(19) United States
(12) Patent Application Publication
(10) Pub. No.: US 2013/0012472 A1
(43) Pub. Date: Jan. 10, 2013

(54) COMPOSITION AND METHODS OF INHIBITING GASTROINTESTINAL PATHOGEN INFECTION

(75) Inventors: David S. NEWBURG, Newtonville, MA (US); Yingying HE, Malden, MA (US)

(73) Assignee: Boston College, Chestnut Hill, MA (US)

(21) Appl. No.: 13/542,302

(22) Filed: Jul. 5, 2012

Related U.S. Application Data
(60) Provisional application No. 61/504,487, filed on Jul. 5, 2011.

(51) Int. Cl. A61K 31/726; C07H 5/06; A61P 1/00; A61P 31/00; A61P 29/00

(52) U.S. Cl. 514/54; 536/55.1

(57) ABSTRACT

Compositions comprising a milk-derived oligosaccharide such as trifucosyl(1,2-1,2-1,3)-lacto-N-octoase (TFiLNO) and uses thereof for inhibiting invasion of gastrointestinal pathogen into intestinal epithelial cells or for treating an infectious disease (e.g., a disease caused by a gastrointestinal pathogen such as an ETEC) or an inflammatory diseases such as inflammatory bowel disease.

### RELATED APPLICATION

This application claims the benefit of U.S. provisional application No. 61/504,487, filed Jul. 5, 2011 under 35 U.S.C. §119, the entire content of which is herein incorporated by reference.

### GOVERNMENT SUPPORT

This invention was made with government support awarded by the National Institutes of Health under Grant Number P01 HD013021 and U01 AI075563. The Government has certain rights in the invention.

### BACKGROUND OF THE INVENTION

Enterotoxigenic Escherichia coli (ETEC) is a major infectious agent in developing countries, causing diarrhea in tens of thousands of children. It is the major cause of mortality in children under the age of 5. Stable and labile toxins of E. coli (STa and LT) are considered the major pathogenic agents of ETEC. Other mechanisms of pathogenesis, e.g., bacterial invasion, have also been suggested. However, the relationship between bacterial invasion and pathogenesis has not been well clarified.

It has been found that nursing infants have lower risk of diarrhea that those artificially fed infants. Human milk oligosaccharides (HMOS) were thought as a very important composition of innate immune systems in infants. It is of great interest to identify specific milk components that possess therapeutic effects.

### SUMMARY OF THE INVENTION

The present disclosure in based on the unexpected discoveries that enterotoxigenic Escherichia coli (ETEC) is capable of invading into intestinal epithelial cells, resulting in inflammation, and human milk oligosaccharides, particularly trifucosyl (1,2-1, 2-1,3)-lacto-N-octoase (TFiLNO) contained therein, effectively inhibited ETEC invasion and reduced inflammation induced by the invasion.

Accordingly, one aspect of the present disclosure features a method for inhibiting invasion of intestinal epithelial cells by a gastrointestinal pathogen (e.g., an ETEC), the method comprising administering (e.g., orally) to a subject in need thereof a synthetic composition that comprises a milk-derived oligosaccharide (e.g., TFiLNO or a fragment thereof), wherein the milk-derived oligosaccharide is in an amount effective to inhibit the invasion. When desired, the amount of the milk-derived oligosaccharide is in an amount effective to reduce inflammation induced by the invasion.

The subject to be treated by the method described herein can be a human subject, e.g., a human child under the age of 5 such as a human infant. In some embodiments, the subject is infected, suspected of being infected, or at risk for infection by the gastrointestinal pathogen (e.g., an ETEC). In other embodiments, the subject is a human having or being suspected of having an inflammatory bowel disease.

The synthetic composition to be used in the method described herein can be a pharmaceutical composition, which further comprises a pharmaceutically acceptable carrier. Alternatively, it can be a nutritional composition or an infant formula. In some examples, the synthetic composition comprises mammalian milk oligosaccharides (MMOS) such as human milk oligosaccharides (HMOS). In other examples, the synthetic composition is substantially free of the MMOS or HMOS.

In another aspect, the present disclosure features a method for treating an infectious disease (e.g., a disease caused by a gastrointestinal pathogen such as an ETEC), or an inflammatory disease such as IBD, the method comprising administering to a subject in need thereof an effective amount of trifucosyl (1,2-1, 2-1,3)-lacto-N-octoase (TFiLNO) or a fragment thereof. The subject can be a human subject as described herein, e.g., a human patient who has, is suspected of having, or is at risk for the infectious or inflammatory disease.

The present disclosure also features a synthetic composition, comprising trifucosyl (1,2-1, 2-1,3)-lacto-N-octoase (TFiLNO) or a fragment thereof, wherein the composition is substantially free of MMOS (e.g., HMOS). Such a synthetic composition can be a pharmaceutical composition that further comprises a pharmaceutically acceptable carrier, a nutritional composition, or an infant formula.

Also within the scope of this disclosure are any of the synthetic compositions described herein for use in treating an infectious disease such as an infection by a gastrointestinal pathogen (e.g., an ETEC) or an intestinal inflammatory disease such as inflammatory bowel disease, and for use in manufacturing a medicament for the treatment of such infectious and inflammatory diseases.

The details of one or more embodiments of the invention are set forth in the description below. Other features or advantages of the present invention will be apparent from the following drawings detailed description of several embodiments, and also from the appended claims.

### BRIEF DESCRIPTION OF THE DRAWINGS

The drawings are first described.

FIG. 1 is a schematic illustration showing the structure of trifucosyl(1,2-1, 2-1,3)-lacto-N-octoase (TFiLNO).

FIG. 2 is a chart showing invasion of an ETEC(H10407) into various types of intestinal epithelial cells (IECs), including HCT8 cells, Hela cells, Caco2 cells, T84 cells, FHs74 cells, and H4 cells, during in vitro infection.

FIG. 3 shows that invasion of ETEC into IECs augmented secretion of various pro-inflammatory factors, which is a LPS-dependent and long term effect. A: a chart showing invasion of ETEC into T84 cells increased the secretion levels of pro-inflammatory factors IL-6, IL-8, TNF-α, and MCP-1. B and G: dosage curves showing that IL-8 secretion induced by ETEC invasion is dose dependent. C: a chart showing alive bacteria are necessary for augmentation of IL-8 secretion. D: a chart showing inhibition of invasion by cytochalasin D led to suppression of IL-8 secretion. E: a chart showing the invasion-induced inflammation is LPS dependent. F: a curve showing the long-term effect of ETEC invasion into T84 cells and induction of IL-8 production.

FIG. 4 shows the inhibitory effects of HMOS on ETEC adherence to IECs (A), ETEC invasion into TECs (B), and inflammation induced by ETEC invasion (C).

FIG. 5 shows the inhibitory effects of TFiLNO on ETEC adherence to IECs (A), ETEC invasion into IECs (B), and inflammation induced by ETEC invasion (C).

FIG. 6 shows that TFiLNO does not kill bacteria; nor does it bind to bacteria in the inhibition process. A: bacteria killing assays showed that TFiLNO did not kill ETEC. B: Invasion of ETEC was not inhibited when ETEC was pre-incubated with TFiLNO. C: Wash assays showed that washing off preincubated TFiLNO did not affect its inhibitory activity against ETEC invasion.

FIG. 7 illustrates that TFiLNO directly inhibited LPS-stimulated IL-8 secretion by suppressing CD14 expression on IECs. A: a chart showing that TFiLNO inhibited IL-8 secretion stimulated by LPS. B: a chart showing the inhibitory effect of TFiLNO in reducing IL-8 secretion is mediated by suppression of CD14 expression on IEC cells. C and D: FACS analysis showing suppression of CD14 expression on TFiLNO-treated IECs.

### DETAILED DESCRIPTION OF THE INVENTION

As the third most abundant solid component of human milk, HMOS are in milk at a concentration of about 5-12 mg per mL, containing over 200 individual oligosaccharides. It has been reported that HMOS are protective reagents in infants against infectious diseases caused by various pathogens. Without being bound by theory, HMOS may play pleiotropic effects by: a) preventing intestinal epithelial surface binding of enteropathogenic bacteria (including Salmonella, ETEC, EPEC, and rotavirus) via mimicking the glycan moieties of receptors on the host cells; and b) serving as prebiotics that promote beneficial bacterial growth at the proper time; and c) modifying the innate immunity system on the mucosal surface. See, e.g., Kunz et al., Annual Review of Nutrition, 2000, 20:699-722; Schwertmann et al., J. Pediatr. Gastroenterol. Nutr. 28:257-263; and Hickey, International Dairy Journal, 2012, 22:141-146. However, little was known on how HMOS directly modify the innate immunity system. It has been hypothesized that HMOS may modulate the infants' immune system and reduce mucosal neutrophil infiltration and activation. In addition, disialyllacto-N-tetraose is suggested to prevent necrotizing enterocolitis in neonatal rats. Jantscher-Krenn et al., Gut, 2011, December 3.

In the present disclosure, an in vitro model involving intestinal epithelial T84 cells was used to study the anti-inflammatory role of HMOS. Unexpectedly, it was found that (a) ETEC invaded IECs, which subsequently induced inflammation, and (b) HMOS, particularly TFiLNO contained therein, inhibited such invasion and the inflammation induced thereby. These results suggest that blocking ETEC invasion into IECs would be an effective approach in treating infection caused by ETEC or other similar gastrointestinal pathogens and that sugar components in milk such as TFiLNO possess such inhibitory effects. Accordingly, the present disclosure relates to synthetic compositions comprising one or more milk-derived oligosaccharides such as TFiLNO or HMOS, and uses thereof for inhibiting invasion of a gastrointestinal pathogen (e.g., ETEC) into IECs and/or treating infection caused thereby, and for treating intestinal inflammatory disease, for example, inflammatory. Further, as low efficiency invasion of IECs by gastrointestinal pathogens is suggested to be an important reason for inflammatory bowel disease (Barnich et al., J. Clin. Invest., 2007, 117:1566-1574), the milk-derived oligosaccharides, such as TFiLNO, can also be used in treating inflammatory diseases of the intestinal mucosa, including inflammatory bowel disease (IBD).

### Milk-derived Oligosaccharides

A milk-derived oligosaccharide has at least three sugar units and is either a naturally-occurring oligosaccharide found in milk, a fragment of the naturally-occurring oligosaccharide, or a variant thereof that contains a modified (e.g., sulfated, acetylated, or phosphorylated) sugar unit as compared to its natural counterpart. Milk-derived oligosaccharides are well known in the art. See, e.g., U.S. Patent Application No. 61/168,674, WO2005/055944, U.S. Pat. No. 7,893,041, and U.S. patent application Ser. No. 13/382,323, all of which are incorporated by reference herein. The following tables list exemplary oligosaccharides that are found in human milk:

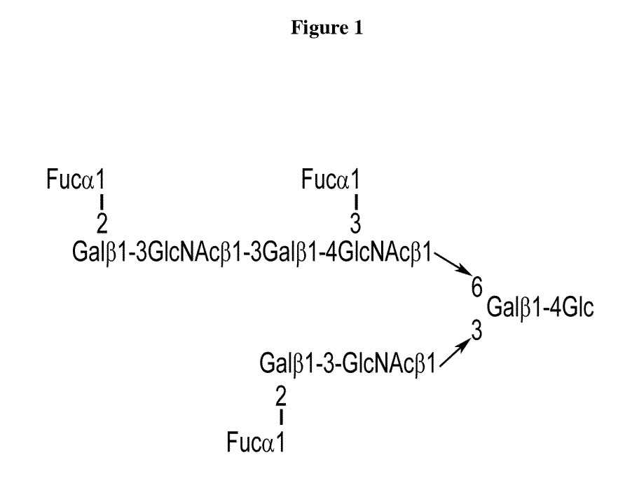

TABLE 1
Fucosyl oligosaccharides

| | | |
|---|---|---|
| 2′FL | 2-Fucosyllactose | Fucα1,2Galβ1,4Glc |
| LNF-I | Lacto-N-fucopentaose I | Fucα1,2Galβ1,3GlcNAcβ1,3Galβ1,4Glc |
| LNF-II | Lacto-N-fucopentaose II | 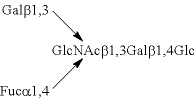 |
| 3′FL | 3-Fucosyllactose | 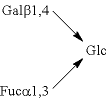 |
| LNF-III | Lacto-N-fucopentaose III | 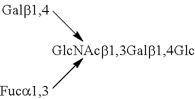 |
| LDFH-I | Lacto-N-difucohexaose I | 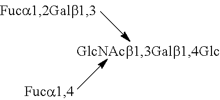 |
| LDFT | Lactodifucotetraose | 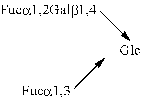 |
| TFiLNO | Trifucosyl(1,2-1,2-1,3)- | See FIG. 1 |
| | lacto-N-octoase | |

TABLE 2
Nonfucosylated, nonsialylated oligosaccharides

| | | |
|---|---|---|
| LNT | Lacto-N-tetraose | Galβ1,3GlcNAcβ1,3Galβ1,4Glc |
| LNneoT | Lacto-N-neotetraose | Galβ1,4GlcNAcβ1,3Galβ1,4Glc |

TABLE 3
Sialyl milk oligosaccharide structures

| | | |
|---|---|---|
| 3′-SL | 3′-Sialyllactose | NANAα2,3Galβ1,4Glc |
| 6′-SL | 6′-Sialyllactose | NANAα2,6Galβ1,4Glc |
| SLNT-c | Sialyllacto-N-neotetraose c | NANAα2,6Galβ1,4GlcNAcβ1,3Galβ1,4Glc |
| MSLNH | Monosialyllacto-N-hexaose | 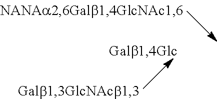 |
| DSLNH-I | Disialyllacto-N-hexaose I | 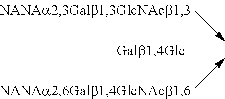 |
| MSLNnH-I | Monosialyllacto-N-neohexaose I | 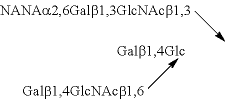 |
| SLNnH-II | Monosialyllacto-N-neohexaose II | 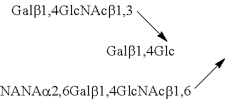 |
| DSLNnH | Disialyllacto-N-neohexaose | 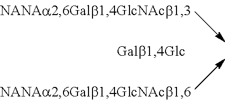 |
| DSLNT | Disialyllacto-N-tetraose | 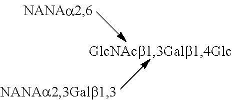 |
| DSLNH-II | Disialyllacto-N-hexaose II | 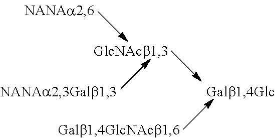 |
| SLNT-a | Sialyllacto-N-tetraose a | NANAα2,3Galβ1,3GlcNAcβ1,3 Galβ1,4Glc |
| DSLNH-I | Disialyllacto-N-hexaose I | 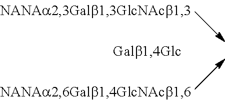 |
| SLNT-b | Sialyllacto-N-tetraose b | 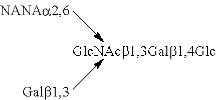 |

TABLE 4
Sialyl fucosyl oligosaccharides

| | | |
|---|---|---|
| 3′-S-3FL | 3′-Sialyl-3-fucosyllactose | 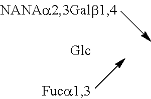 |
| DSFLNH | Disialomonofucosyllacto-N-neohexaose | 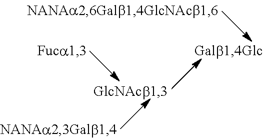 |
| MFMSLNO | Monofucosylmonosialyllacto-N-octaose (sialyl Lea) | 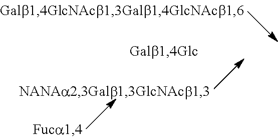 |
| SLNFH-II | Sialyllacto-N-fucohexaose II | 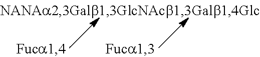 |
| DSLNFP-II | Disialyllacto-N-fucopentaose II | 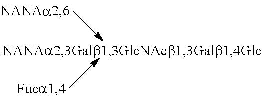 |
| MFDLNT | Monofucosyldisialyllacto-N-tetraose | 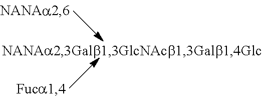 |

In one embodiment, a milk-derived oligosaccharide is trifucosyl(1,2-1,2,-1,3)-lacto-N-octoase (TFiLNO), an oligosaccharide found in human milk. The term “trifucosyl(1,2-1,2,-1,3)-lacto-N-octoase” or “TFiLNO” used herein refers to either the naturally-occurring oligosaccharide having the structure shown in FIG. 1, or a modified variant thereof (e.g., having a sulfated, acetylated, or phosphorylated sugar unit as compared to its natural counterpart). In addition to TFiLNO, a functional fragment thereof (a fragment of the oligosaccharide that preserves at least 50%, e.g., 60%, 70%, 80%, 90%, or 95%, of the bioactivity of TFiLNO such as the inhibitory activities described herein) is also within the scope of this disclosure.

The milk-derived oligosaccharides described herein can be prepared by conventional methods, e.g., synthesized chemically, purified from milk, or produced in a microorganism. See WO2005/055944 and U.S. Pat. No. 7,893,041. For example, TFiLNO can be isolated from human milk via conventional purification methods, e.g., ultrafiltration, microfiltration, HPLC, FPLC, affinity chromatography, and paper chromatography. See, e.g., Strecker et al., Carbohydrate Research, 226:1-14 (1992) and Strecker et al., Glycoconjugate J. 6:67-83 (1989). In one example, TFiLNO can be isolated as follows. Fractions V, VI, and VIII, obtained by preparative paper chromatography (Streeker et al., Glycoconjugate J., 5 (1988) 385-396.) can be respectively fractionated into 8, 3, and 17 peaks on a 5-q ODS Zorbax column (25×0.95 cm), with water as eluent. Oligosaccharide compounds V-1,2 and V-3,5 can be recycled two times on the same column, until total purification. Finally, oligosaccharide compounds V-1,2, V-3,5, VI-2, and VIII-16,17 can be obtained from combined samples of milk. Two h.p.l.c. peaks can be obtained for each oligosaccharide, which correspond to the p (first peak) and the a anomer (second peak) of the compound. The oligosaccharides thus obtained can be subjected to structural analysis to confirm that their identification.

In other embodiments, the milk-derived oligosaccharides described herein comprise mammalian milk oligosaccharides (MMOS), such as human milk oligosaccharides (HMOS). MMOS or HMOS refers to a collection of the sugar components of mammalian milk such as human milk. Such a sugar collection can be purified from mammalian milk via routine procedures. Below is an example:

Mammalian milk is first defatted by centrifugation to produce skimmed milk. The skimmed milk is then mixed with an organic solvent, such as acetone (e.g., 50% aqueous acetone) or ethanol (e.g., 67% aqueous ethanol), to precipitate milk proteins. Upon centrifugation, the supernatant is collected and subjected to chromatography. Oligosaccharide-containing fractions are collected and pooled. If necessary, the oligosaccharides thus prepared can be concentrated by conventional methods, e.g., dialysis or freeze-drying.

In another example, milk oligosaccharides can also be isolated from skimmed milk by passing the skimmed milk through a 30,000 MWCO ultrafiltration membrane, collecting the diffusate, passing the diffusate through a 500 MWCO ultrafilter, and collecting the retentate, which contains milk oligosaccharides.

When necessary, any of the milk-derived oligosaccharides as described herein (e.g., TFiLNO or a fragment thereof) can be linked to a backbone molecule, such as a polypeptide, a lipid, a carbohydrate, or a nucleic acid, to form a glycoconjugate. A glycoconjugate can be a complex containing a sugar moiety associated with a backbone moiety. The sugar and the backbone moieties can be associated via a covalent or noncovalent bond, or via other forms of association, such as entrapment (e.g., of one moiety on or within the other, or of either or both entities on or within a third moiety). The glycoconjugate described herein can contain one type of milk-derived oligosaccharide (i.e., one or more copies of a milk-derived oligosaccharide attached to one backbone molecule). Alternatively, the glycoconjugate contains multiple types of milk-derived oligosaccharides. In one example, the milk-derived oligosaccharide as described herein is covalently linked via its reducing end sugar unit to a lipid, a protein, a nucleic acid, or a polysaccharide. Preferably, the reducing end sugar unit is N-acetylglucosamine.

Peptide backbones suitable for making the glycoconjugate described above include those having multiple glycosylation sites (e.g., asparagine, lysine, serine, or threonine residue) and low allergenic potential. Examples include, but are not limited to, amylase, bile salt-stimulated lipase, casein, folate-binding protein, globulin, gluten, haptocorrin, lactalbumin, lactoferrin, lactoperoxidase, lipoprotein lipase, lysozyme, mucin, ovalbumin, and serum albumin.

Typically, a milk-derived oligosaccharide can be covalently attached to a serine or threonine residue via an O-linkage or attached to an asparagine residue via an N-linkage. To form these linkages, the sugar unit at the reducing end of the oligosaccharide is preferably an acetylated sugar unit, e.g., N-acetylgalactosamine, N-acetylglucosamine, and N-acetylmannosamine. An oligosaccharide can be attached to a peptide (e.g., a protein) using standard methods. See, e.g., McBroom et al., Complex Carbohydrates, Part B, 28:212-219, 1972; Yariv et al., Biochem J., 85:383-388, 1962; Rosenfeld et al., Carbohydr. Res., 46:155-158, 1976; and Pazur, Adv. Carbohydr. Chem., Biochem., 39:405-447, 1981.

In one example, a milk-derived oligosaccharide is linked to a backbone molecule via a linker. Exemplary linkers are described in WO2005/055944 and U.S. Pat. No. 7,893,041. The oligosaccharide can be bonded to a linker by an enzymatic reaction, e.g., a glycosyltransferase reaction. A number of glycosyltransferases, including fucosyltransferases, galactosyltransferases, glucosyltransferases, mannosyltransferases, galactosaminyltransferases, sialyltransferases and N-acetylglucosaminyltransferases, can be used to make the glycoconjugate described herein. More details about these glycosyltransferases can be found in U.S. Pat. Nos. 6,291,219; 6,270,987; 6,238,894; 6,204,431; 6,143,868; 6,087,143; 6,054,309; 6,027,928; 6,025,174; 6,025,173; 5,955,282; 5,945,322; 5,922,540; 5,892,070; 5,876,714; 5,874,261; 5,871,983; 5,861,293; 5,859,334; 5,858,752; 5,856,159; and 5,545,553.

### Synthetic Compositions

Any of the milk-derived oligosaccharides described herein (e.g., TFiLNO or a fragment thereof, or MMOS such as HMOS) can be formulated to produce synthetic compositions, which refer to non-naturally occurring compositions. While the synthetic compositions described herein may contain components (e.g., oligosaccharides) found in milk, they are not milk products (e.g., raw milk, homo milk, or skimmed/defatted milk) or fermented milk products (also known as cultured dairy foods, cultured dairy products, or cultured milk products), such as cheese, buttermilk, or yogurt. The synthetic composition described herein can contain ingredients that are purified or isolated or are otherwise artificially (not naturally) synthesized. The sugar content of such a synthetic composition, including the varieties of oligosaccharides and the relative amount of each oligosaccharide as compared to the total sugar component in the composition, may differ from that in mammalian milk such as human milk. In one example, such a synthetic composition is substantially free of any MMOS, such as HMOS, e.g., containing less than 10% (5%, 2%, or 1%) by weight of MMOS.

In some examples, the specified active ingredients (e.g., the milk-derived oligosaccharide such as TFiLNO or HMOS) constitute at least about 25%, 30%, 35%, 40%, 50%, 60%, 70%, 80%, 90%, or 95% of the respective composition by weight. In other examples, the specified oligosaccharide(s) constitute at least about 30%, 40%, 50%, 60%, 70%, 80%, 90%, or 95% of the total sugar content in the composition by weight. In yet other examples, the weight percentage of the specified milk-derived oligosaccharide in the total sugar content of the composition is at least 2-fold, 3-fold, 5-fold, 10-fold, or 100-fold higher than the weight percentage of the same oligosaccharide in the total sugar content in mammalian milk, such as in human milk. When necessary, the synthetic composition described herein comprises TFiLNO and one or more other oligosaccharides found in human milk, such as 2′-FL, 3-FL, and others listed in the tables above.

The synthetic compositions described herein can be formulated and administered in any suitable form known to those skilled in the art. For enteral administration, the compositions can be formulated into preparations in solid, semi-solid, gel, or liquid forms such as tablets, capsules, powders, granules, solutions, depositories, gels, and injections. Compositions suitable for oral administration may be presented as discrete units, such as capsules, tablets, lozenges, each containing a predetermined amount of an active agent. Other compositions include suspensions in aqueous liquids or non-aqueous liquids such as a syrup, elixir, gels, or emulsions. They may also be in pre-weighted packets of power, such as sachets.

In some examples, the synthetic compositions described herein are nutritional compositions, which represent a food composition or a food supplement that does not possess the characteristics of a drug. The nutritional composition can be an infant formula, which is a food product designed for feeding to babies and infants, e.g., under 12 months of age. Such nutritional compositions can be prepared following routine procedures in the food industry.

In other examples, the synthetic composition is a pharmaceutical composition, which further comprises a pharmaceutically acceptable carrier. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, lactose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In other examples, the pharmaceutical composition described herein can be formulated in sustained-release format. Suitable examples of sustained-release preparations include semipermeable matrices of solid hydrophobic polymers containing the oligosaccharide, which matrices are in the form of shaped articles, e.g. films, or microcapsules. Examples of sustained-release matrices include polyesters, hydrogels (for example, poly(2-hydroxyethyl-methacrylate), or poly(v nylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic compositions are generally placed into a container having a sterile access port, for example, an intravenous solution bag or vial having a stopper pierceble by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g. Tween™ 20, 40, 60, 80 or 85) and other sorbitans (e.g. Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g. soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0. im, particularly 0.1 and 0.5. im, and have a pH in the range of 5.5 to 8.0.

The emulsion compositions can be those prepared by mixing a milk-derived oligosaccharide with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulised by use of gases. Nebulised solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

### Uses of Milk-Derived Oligosaccharides in Treating Infectious or Inflammatory Diseases

To practice the method disclosed herein, an effective amount of the pharmaceutical composition described above can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers, are useful for administration. Liquid formulations can be directly nebulized and lyophilized powder can be nebulized after reconstitution. Alternatively, compositions comprising any of the milk-derived oligosaccharides as described herein can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having been infected with a gastrointestinal pathogen, such as an ETEC. In some embodiments, the human subject has, is suspected of having, or at risk for an intestinal inflammatory disease, such as inflammatory bowel disease (IBD), including ulcerative colitis or Crohn's disease, which involves chronic inflammation of all or part of the digestive tract. A subject suffering from infection of a gastrointestinal infection or an inflammatory disease such as IBD can be identified via routine medical practices.

A subject suspected of having, or being at risk for a disease refers to a subject having an elevated level of suspicion of the presence of the disease or an elevated level of risk for contracting the disease, as compared to an average level of suspicion for average risk level. For example, a subject manifesting clinical symptoms of a specific disease has an elevated level of suspicion of the presence of the disease, even in the absence of an objective clinical diagnosis. For another example, the subject may be predisposed to contracting a specific disease, for example, because of the subject's genetic makeup, or because of exposure to environmental pathogens, or because of the presence of behavioral risk factors, such as dietary or other behavioral habits.

In some examples, the subject to be treated by any of the methods described herein is a human child, e.g., a child under the age of five. In other examples, the subject is a human infant, e.g., under the age of 12 months. In yet other examples, the subject is a human adult, e.g., a human elder such as a person over the age of 55.

An effective amount can refer to the amount of each active agent required to confer a desired therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of the infectious or inflammatory disease. Alternatively, sustained continuous release formulations of the milk-derived oligosaccharide may be appropriate. Various formulations and devices for achieving sustained release are known in the art.

In one example, dosages for a milk-derived oligosaccharide such as TFiLNO as described herein may be determined empirically in individuals who have been given one or more administration(s) of the oligosaccharide. Individuals are given incremental dosages of the oligosaccharide. To assess efficacy of the oligosaccharide, an indicator of infection or inflammation, e.g., the level of a pro-inflammatory factor like IL-8, can be followed.

The milk-derived oligosaccharide may be administered at the rate of about 0.1 to 300 mg/kg of the weight of the patient divided into one to three doses, or as disclosed herein. In some embodiments, for an adult patient of normal weight, doses ranging from about 0.3 to 5.00 mg/kg may be administered. The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate liver fibrosis or cirrhosis, or a symptom thereof. An exemplary dosing regimen comprises administering an initial dose of about 2 mg/kg, followed by a weekly maintenance dose of about 1 mg/kg of the oligosaccharide, or followed by a maintenance dose of about 1 mg/kg every other week. In some examples, the dosage of the synthetic composition is designed such that the intestinal concentration of the milk-derived oligosaccharide is close to that in human milk (e.g., 30 pg/mL when TFiLNO is used). However, other dosage regimens may be useful, depending on the pattern of pharmacokinetic decay that the practitioner wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the oligosaccharide used) can vary over time.

In some embodiments, any of the synthetic compositions as described herein is used to inhibit invasion of a gastrointestinal pathogen (e.g., an ETEC) into intestinal epithelial cells in a subject and/or alleviate the inflammation caused by the invasion. Gastrointestinal pathogens include pathogens such as bacteria that can colonize in the gut of a subject and cause and/or do cause a disease or condition in the subject. Exemplary gastrointestinal pathogens include, but are not limited to Escherichia coli, Clostridium perfringens, Listeria monocytogenes, Listeria innocua, Staphylococcus aureus, Enterococcus faecalis (virulent strains of E. faecalis), and Enterococcus faecium.

The amount in such a synthetic composition can be effective to inhibit the invasion or alleviate the inflammation caused thereby. Inhibiting invasion refers to decreasing the rate of pathogen invasion into IECs when contacted with the pathogen and/or the IECs. In one example, the amount of the milk-derived oligosaccharide used in the method described above can reduce the rate of pathogen invasion into IECs by at least 20%, 40%, 60%, 80%, 1-fold, 2-fold, 5-fold, 10-fold, 50-fold, 100-fold, or 200-fold, as compared to the rate in the absence of the oligosaccharide.

Alleviating inflammation refers to reduction of the level of intestinal inflammation stimulated by pathogen infection/invasion. In some examples, the amount of the milk-derived oligosaccharide used in the method described above can reduce the intestinal inflammation level by at least 20%, 40%, 60%, 80%, 1-fold, 2-fold, 5-fold, 10-fold, 50-fold, 100-fold, or 200-fold, as compared to the inflammation level in the absence of the oligosaccharide.

In other embodiments, any of the synthetic compositions as described herein is used for treating an intestinal infectious or inflammatory disease such as IBD. In one example, such a synthetic composition comprises TFiLNO or a fragment thereof, and optionally, one or more additional milk-derived oligosaccharides. The term “treating” as used herein refers to the application or administration of a composition including one or more active agents to a subject in who has any of the infectious or inflammatory diseases described herein, a symptom of the disease, or a predisposition toward the disease, with the purpose to cure, heal, alleviate, relieve, alter, remedy, ameliorate, improve, or affect the disease, the symptoms of the disease, or the predisposition toward the disease.

Conventional methods, known to those of ordinary skill in the art of medicine, can be used to administer the synthetic compositions described herein to the subject, depending upon the type of disease to be treated. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods.

Injectable compositions may contain various carriers such as vegetable oils, dimethylactamide, dimethylormamide, ethyl lactate, ethyl carbonate, isopropyl myristate, ethanol, and polyols (glycerol, propylene glycol, liquid polyethylene glycol, and the like). For intravenous injection, water-soluble antibodies can be administered by the drip method, whereby a pharmaceutical formulation containing the milk-derived oligosaccharide and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the oligosaccharide, can be dissolved and administered in a pharmaceutical excipient such as Water-for-Injection, 0.9% saline, or 5% glucose solution.

For oral administration, a milk-derived oligosaccharide such as TFiLNO can be formulated readily by combining with pharmaceutically acceptable carriers or edible carriers well known in the art. Such carriers enable an active agent to be formulated as tablets, pills, dragees, capsules, liquids, gels, syrups, slurries, suspensions and the like, for oral ingestion by a subject to be treated. Pharmaceutical or nutritional preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical or nutritional preparations which can be used orally include push fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such micro spheres have been well defined in the art. All formulations for oral administration should be in dosages suitable for such administration.

Kits For Use in Inhibiting Pathogen Invasion into IECs or Alleviating Inflammation

The present disclosure also provides kits for use in inhibiting invasion of an intestinal pathogen (e.g., an ETEC) into IECs or alleviating inflammation caused by the invasion. Such kits can include any of the synthetic compositions described herein, which comprise one or more milk-derived oligosaccharides (e.g., TFiLNO or HMOS).

When necessary, the kit can comprise instructions for use in accordance with any of the methods described herein. The included instructions can comprise a description of administration of the oligosaccharides to inhibit pathogen invasion or alleviating inflammation according to any of the methods described herein. The kit may further comprise a description of selecting an individual suitable for any of the treatments based on identifying whether that individual has a target disease or is suspected of having such. In still other embodiments, the instructions comprise a description of administering the oligosaccharide-containing composition to an individual at risk of infection with a gastrointestinal pathogen.

The instructions relating to the use of a milk-derived oligosaccharide generally include information as to dosage, dosing schedule, and route of administration for the intended treatment. The containers may be unit doses, bulk packages (e.g., multi-dose packages) or sub-unit doses. Instructions supplied in the kits as described herein are typically written instructions on a label or package insert (e.g., a paper sheet included in the kit), but machine-readable instructions (e.g., instructions carried on a magnetic or optical storage disk) are also acceptable.

The label or package insert indicates that the composition is used for an intended treatment. Instructions may be provided for practicing any of the methods described herein.

The kits described herein are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle).

Kits may optionally provide additional components such as buffers and interpretive information. Normally, the kit comprises a container and a label or package insert(s) on or associated with the container. In some embodiments, the invention provides articles of manufacture comprising contents of the kits described above.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Examples

### Methods

### Bacteria Invasion Assay

Intestinal epithelial cells (IECs) such as HCT8, T84, Caco2, H4, and FHs74 cells, were cultured in 24-well tissue culture plates (Corning life Sciences Inc, MA) at 5×10⁴ cells/well in antibiotic free media for 48 hours. The IEC cells, with or without treatment of HMOS or TFiLNO, were incubated with a suspension of ETEC H10407 cells (10⁵-10⁹ cfu) for 1 h at 37° C. 50 mg/ml gentamicin was then added to the culture to kill any extracellular bacteria cells. Cell lysis was performed in 500 μl of 0.5% Triton X-100 (Sigma, Mo.) for 30 minutes at room temperature. The cell lysates were plated on difco agar plates (antibiotic free) at a proper dilution of 500. The total number of cell-associated bacteria was determined as the number of bacteria in IEC lysate immediately after infection. The number of invaded bacteria was determined as those detected in IEC cell lysate prepared from IECs treated with gentamycin for 1 hour after bacterial infection. The number of bacteria adhered to IEC cells was calculated as follows: (The total number of cell-associated bacteria)−(The number of invaded bacteria). All of the adhesion and invasion assays were performed in duplicate in at least three independent experiments.

### ELISA

Levels of proinflammatory factors (IL-8, IL-6, TNF-α, and MCP-1) in the culture supernatants of the IECs were measured using ELISA kits from R&D following manufacturer's protocols.

### Preparation of Human Milk Oligosaccharides (HMOS)

Human milk oligosaccharides were prepared from human milk following routine methods (e.g., Chaturvedi et al., Anal. Biochem. 251(1):89-97, 1997). See also U.S. patent application Ser. No. 13/382,323, the entire content of which is incorporated by reference herein. Briefly, pooled human milk was first defatted and then ethanol was added to precipitate proteins. The resultant solution was loaded onto a carbon column, which adsorbs oligosaccharides. The column was washed with 5% ethanol and the adsorbed oligosaccharides were eluted with 60% ethanol to produce a fraction containing human milk oligosaccharides.

### RT-PCR

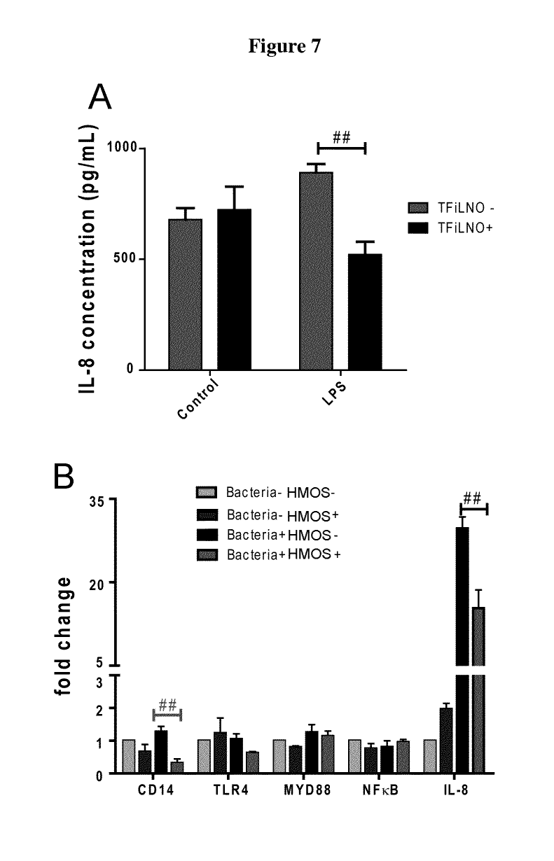

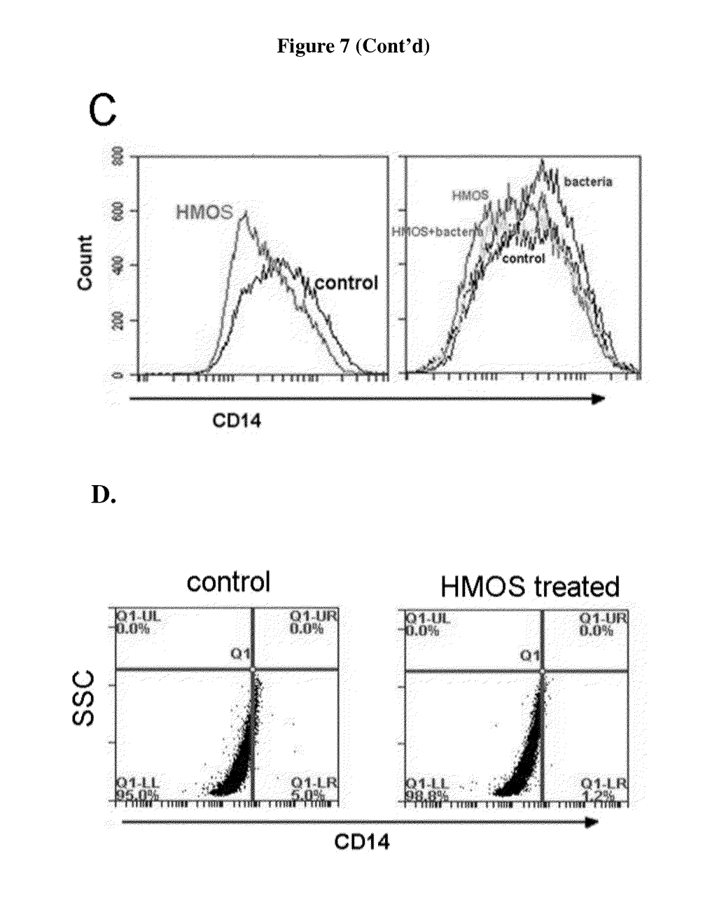

IEC cells (e.g., T84 cells) were treated with HMOS (5 mg/mL) prepared as described above for 48 hours. Cells not treated by HMOS were used as a negative control. Total RNAs were purified from the IEC cells and RT-PCR was performed following routine methods to determine the expression levels of genes of interest, including those shown in FIG. 7B. GADPH was used as an internal control. The relative gene expression levels were determined by Delta CT Method.

### FACS Analysis

T84 cells treated with or without HMOS were incubated with PE-conjugated mouse anti-human CD14 Mabs. PE-conjugated isotype-matched (IgG1) antibodies were used as controls. Data respecting 20,000 live cells from each sample were collected and subjected to FACS analysis following routine procedures.

### Pre-Incubation Assay

ETEC cells in mid-logarithmic growth phase were re-suspended in PBS (pH 7.2) at a concentration of 2×10⁸ cells/ml. The suspension was mixed with an equal volume of PBS in the presence or absence of TFiLNO (30 pg/mL), and incubated in U-bottom 96-well plates at room temperature for 2 h with brief mixing every 15 min. At the end of the incubation, the ETEC cells were co-incubated with T84 cells for 1 hour at 37° C. After extracellular bacteria were killed by gentamycin, invaded ETEC were measured as described above.

### Wash-Out Experiments

IECs were grown in 24-well tissue culture plates (Corning life Sciences Inc, MA) at 5×10⁴ cells/well in an antibiotic free medium for 48 hours. The culture medium was then replaced with a fresh medium containing 30 pg/mL TFiLNO. The IECs were further incubated at 37° C. for 48 hours. Supernatants were discarded and cells were washed 6 times with PBS. The cells were then incubated in a fresh medium free of TFiLNO and contains ETEC (10⁸). After 1 hour incubation, extracellular bacteria were killed by gentamycin. Invaded ETEC were measured as described previously.

### Results

Enterotoxigenic Escherichia Coli (ETEC) Invaded into Intestinal Epithelial Cells (IECs) and Induced Inflammation

(i) ETEC Invades IEC Cells

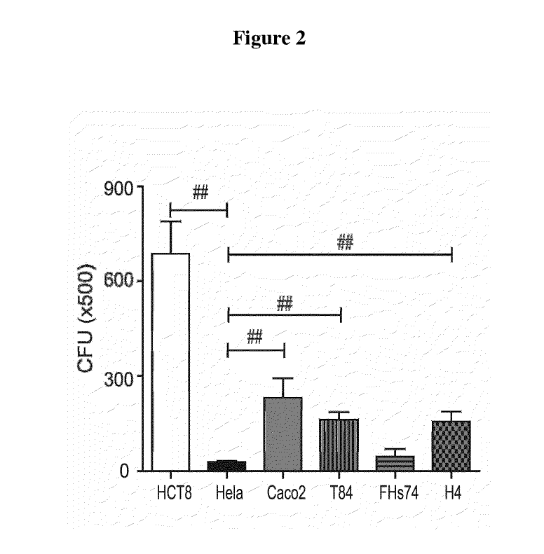

As shown in FIG. 2, ETEC cells (H10407 strain) were found to invade into various types of IEC cells, including HCT, Caco2, T84, H4, and FHs74 cells in the bacteria invasion assay described above. This result was confirmed by Microscopy observation.

(ii) ETEC Invasion of IEC Cells Induced Inflammation

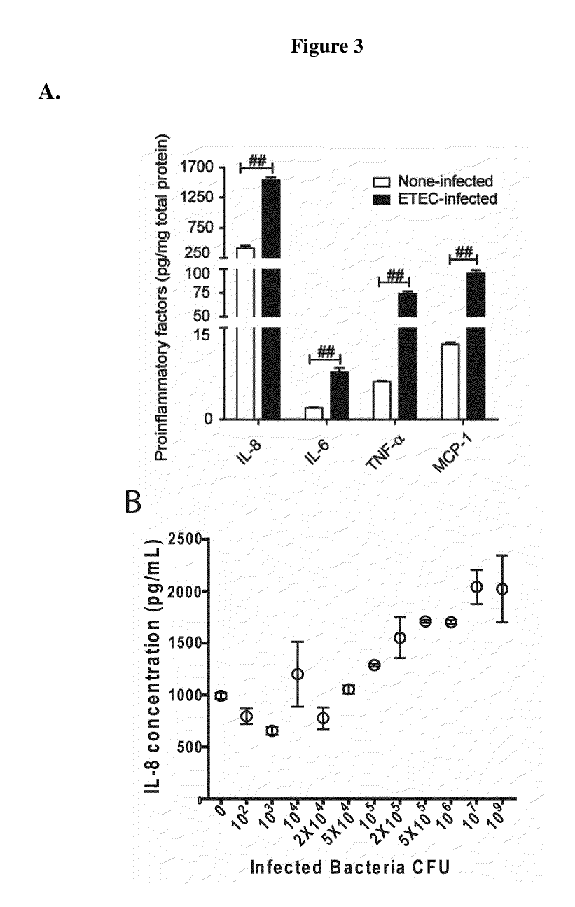

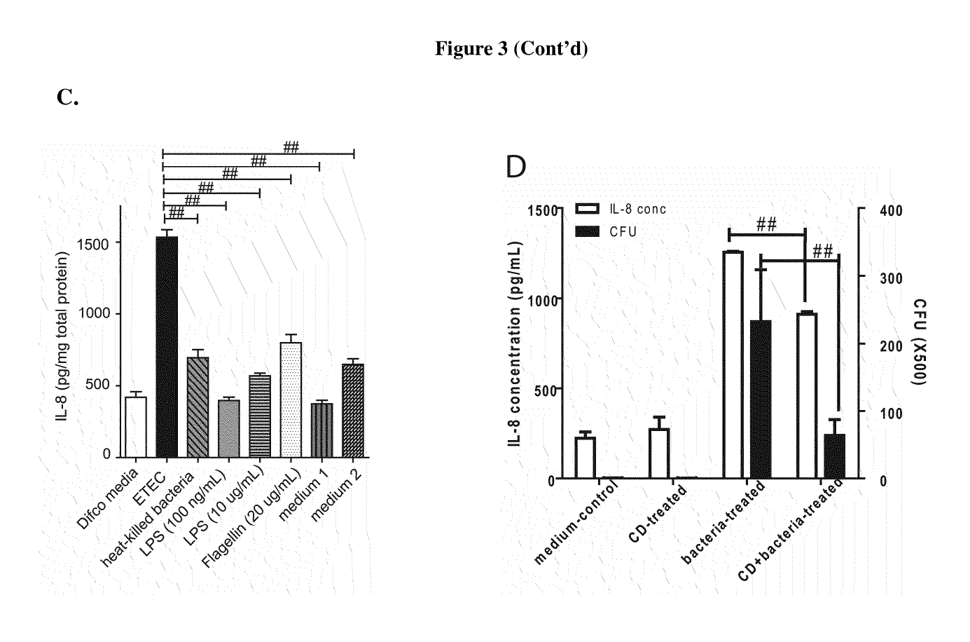

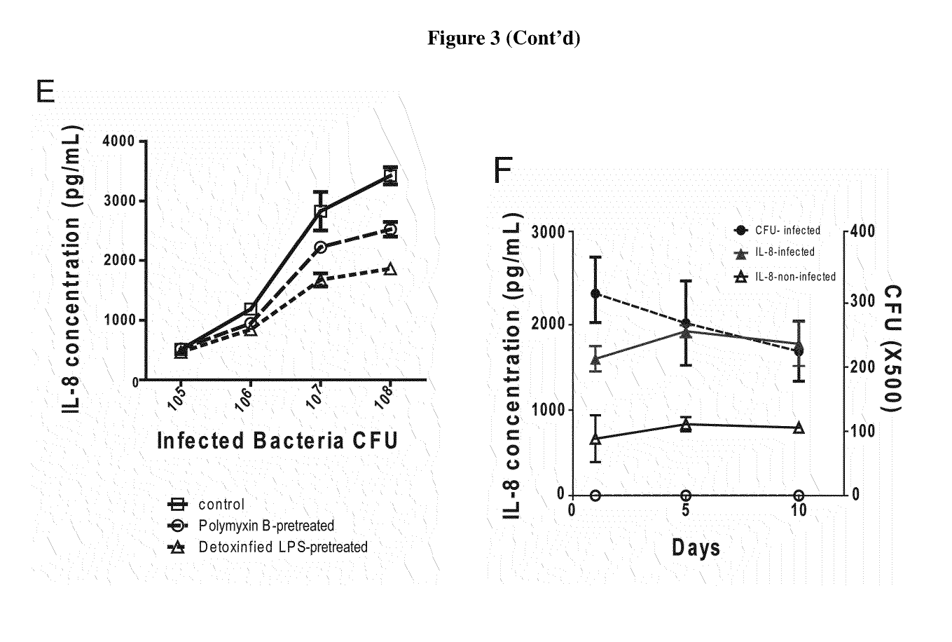

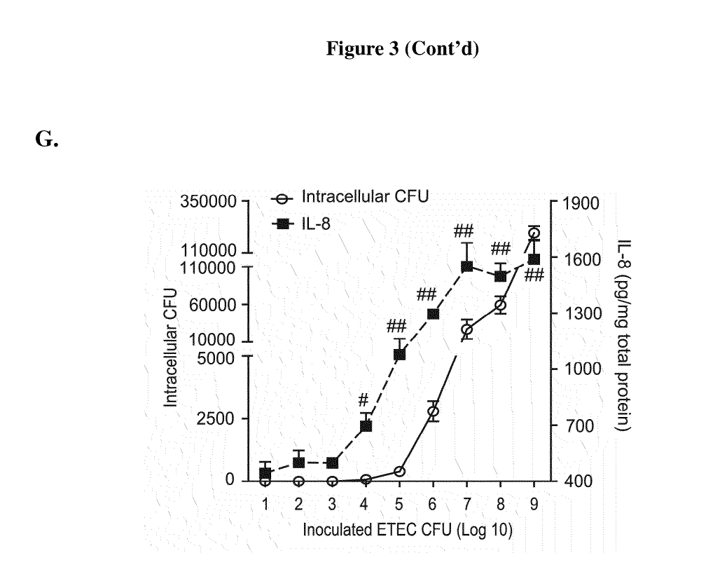

Production of pro-inflammatory factors, including IL-8, IL-6, TNF-α, and MCP-1, by the infected IEC cells was determined by ELISA. Results obtained from this experiment show that ETEC invasion led to a much higher level of secretion of the pro-inflammatory factors into the culture medium as compared to the control cells (not infected with the bacterila). FIG. 3A. The ETEC-induced IL-8 secretion is dose-dependent. FIGS. 3B and 3G. As shown in FIG. 3C, heat-killed bacteria showed significantly reduced ability to induce IL-8 secretion, indicating that live bacteria are necessary for stimulating IL-8 secretion. Inhibition of invasion by cytochalasin D also inhibited IL-8 secretion, confirming that the over-production of IL-8 is induced by bacterial invasion. FIG. 3D. The levels of IL-8 secretion were reduced by polymycin-B and detoxified LPS, indicating that ETEC invasion-induced inflammation was LPS dependent. FIG. 3E. The level of IL-8 secretion in IEC cells invaded by ETEC was higher than that in non-infected cells 10 days after infection, indicating that ETEC invasion of IEC cells (T84 cells) and induction of pro-inflammatory factors were long term effects.

Taken together, the above results indicate that ETEC can invade into IECs, leading to inflammation. Accordingly, blocking ETEC invasion would be an effective approach to alleviate inflammation caused by bacterial infection and/or treating bacterial infection.

### Human Milk Oligosaccharides Inhibits ETEC Invasion and Inflammation

(i) Inhibitory Effects of HMOS on ETEC Adherence, Invasion and ETEC-Induced Inflammation

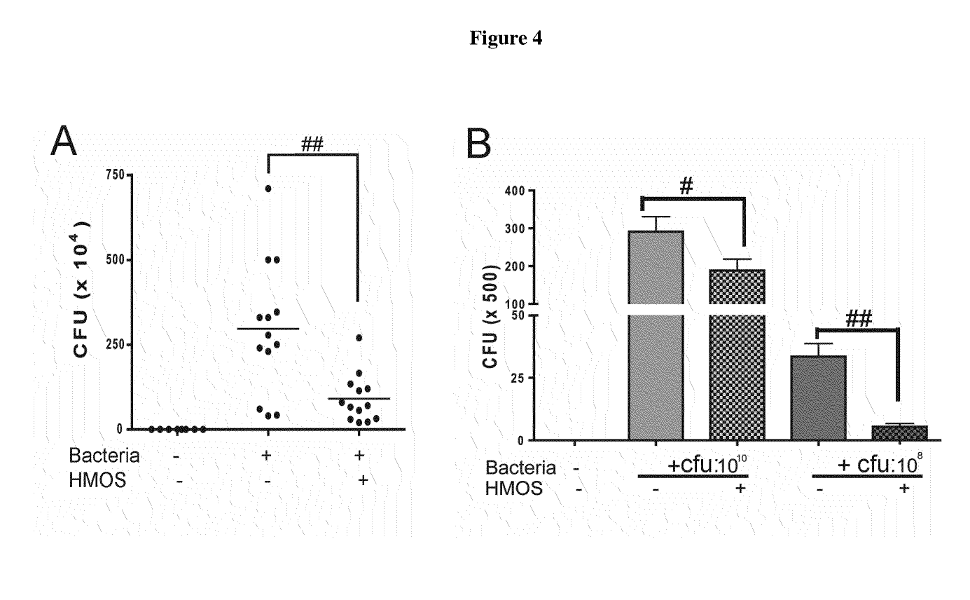

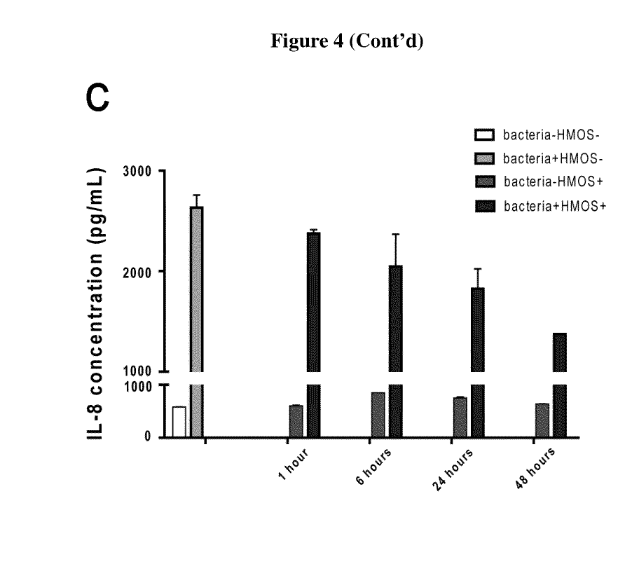

HMOS prepared as described above was used to determine its effects on ETEC infection. As shown in FIGS. 4A and 4B, HMOS successfully reduced ETEC adherence and invasion into IEC cells. Further, secretion of IL-8 by the treated IEC cells was also significantly reduced over time. FIG. 4C. These results indicate that HMOS is effective in reducing ETEC invasion and the inflammation induced thereby.

(ii) Inhibitory Effects of TFiLNO on ETEC Adherence, Invasion and ETEC-Induced Inflammation

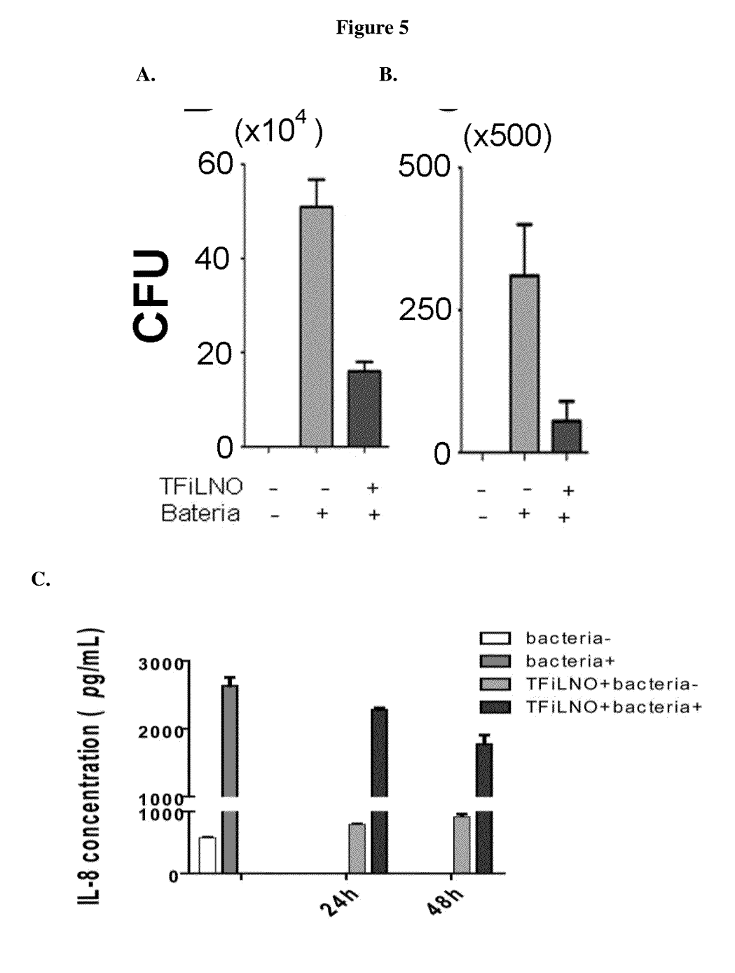

TFiLNO was isolated from human milk following methods known in the art. See, e.g., Strecker et al., Glycoconj. J. 6(1):67-83 (1989). This oligosaccharide (at a concentration of 30 pg/ml) was found to inhibit adherence and invasion of ETEC to IEC cells. FIGS. 5A and 5B. Further, secretion of IL-8 by the treated IEC cells was also significantly reduced over time. FIG. 5C. These results indicate that TFiLNO is an effective agent in reducing ETEC invasion and the inflammation induced thereby.

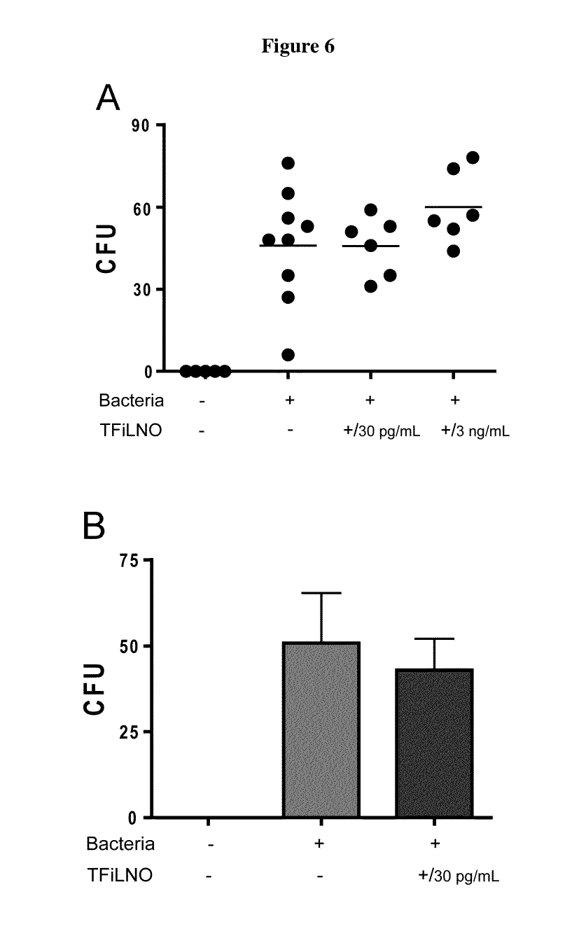

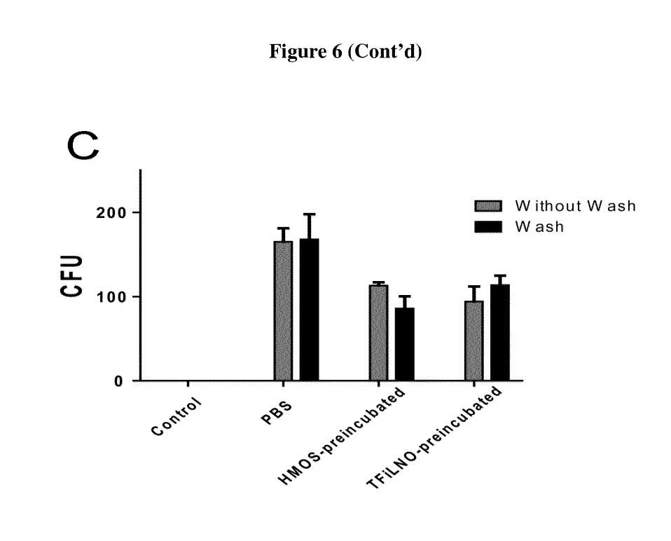

TFiLNO at a concentration of 30 pg/ml did not kill ETEC. FIG. 6A. Invasion of ETEC could not be further inhibited when ETEC was pre-incubated with TFiLNO, indicating that this oligosaccharide did not bind to the bacteria and its inhibitory effect on bacterial invasion is not through its binding to the bacteria. FIG. 6B. This is confirmed by demonstrating that washing off preincubated TFiLNO did not affect its inhibitory activity against ETEC invasion. FIG. 6C.

To examine the effect of TFiLNO on IL-8 production, this oligosaccharide (30 pg/ml) was incubated with T84 cells in the absence of ETEC for 1 h at 37° C. IL-8 secretion to the culture medium was determined by ELISA. As shown in FIG. 7A, TFiLNO directly inhibited IL-8 secretion stimulated by LPS via suppression of CD14 expression on IECs. See also FIGS. 7B and 7C.

In sum, the results obtained from this study show that (a) ETEC can invade IECs during infection, thereby stimulating section of pro-inflammatory factors; (b) this effect is LPS-dependent and long term; (c) HMOS inhibited ETEC adherence, invasion, and the resulting inflammation by suppressing CD14 expression; and (d) TFiLNO may play important roles in this inhibitory process.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method for inhibiting invasion of intestinal epithelial cells by a gastrointestinal pathogen, the method comprising administering to a subject in need thereof a synthetic composition that comprises a milk-derived oligosaccharide, wherein the milk-derived oligosaccharide is in an amount effective to inhibit the invasion.

2. The method of claim 1, wherein the milk-derived oligosaccharide is trifucosyl (1,2-1, 2-1,3)-lacto-N-octoase (TFiLNO) or a fragment thereof.

3. The method of claim 1, wherein the composition comprises mammalian milk oligosaccharides (MMOS).

4. The method of claim 3, wherein the MMOS is human milk oligosaccharides (HMOS).

5. The method of claim 1, wherein the gastrointestinal pathogen is an Enterotoxigenic Escherichia coli (ETEC).

6. The method of claim 1, wherein the subject is a human subject who is infected, suspected of being infected, or at risk for infection by the gastrointestinal pathogen.

7. The method of claim 6, wherein the human subject is a child under 5.

8. The method of claim 7, wherein the human subject is an infant.

9. The method of claim 6, wherein the human subject has or is suspected of having an inflammatory bowel disease.

10. The method of claim 1, wherein the synthetic composition is administered orally.

11. The method of claim 1, wherein the milk-derived oligosaccharide is in an amount effective to reduce inflammation induced by the invasion.

12. The method of claim 1, wherein the synthetic composition is a pharmaceutical composition, which further comprises a pharmaceutically acceptable carrier.

13. The method of claim 1, wherein the synthetic composition is a nutritional composition.

14. The method of claim 1, wherein the synthetic composition is an infant formula.

15. A method for treating an infectious or inflammatory disease, comprising administering to a subject in need thereof an effective amount of trifucosyl (1,2-1, 2-1,3)-lacto-N-octoase (TFiLNO).

16. The method of claim 15, wherein the infectious disease is caused by a gastrointestinal pathogen.

17. The method of claim 16, wherein the gastrointestinal pathogen is an ETEC.

18. The method of claim 15, wherein the subject is a human subject who is infected, suspected of being infected, or at risk for the infectious or inflammatory disease.

19. The method of claim 15, wherein the inflammatory disease is IBD.

20. A synthetic composition, comprising trifucosyl (1,2-1, 2-1,3)-lacto-N-octoase (TFiLNO) or a fragment thereof, wherein the composition is substantially free of MMOS.

21. The synthetic composition of claim 20, wherein the composition is a pharmaceutical composition, which further comprises a pharmaceutically acceptable carrier.

22. The synthetic composition of claim 20, wherein the composition is a nutritional composition.

23. The synthetic composition of claim 20, wherein the composition is an infant formula.
